(19) United States
(12) Patent Application Publication
(10) Pub. No.: US 2025/0247263 A1
(43) Pub. Date: Jul. 31, 2025

(54) SYSTEM AND APPARATUS FOR REMOTE MONITORING AND COMMUNICATION

(71) Applicant: Artisight, Inc., Chicago, IL (US)

(72) Inventors: Andrew Louis Gostine, Chicago, IL (US); Garrett Sterling Larance, Chicago, IL (US); Timothy Robert Koby, Chicago, IL (US)

(73) Assignee: Artsight, Inc., Chicago, IL (US)

(21) Appl. No.: 19/041,815

(22) Filed: Jan. 30, 2025

Related U.S. Application Data
(60) Provisional application No. 63/627,402, filed on Jan. 31, 2024.

(51) Int. Cl. H04L 12/18; H04L 12/10

(52) U.S. Cl. CPC H04L 12/1813; H04L 12/10

(57) ABSTRACT

Remote communication devices, systems, and methods for facilitating communication between remote users is described herein. The remote communication devices may be installed in any suitable location. For example, the remote communication devices and systems may be installed in a medical environment to enable one or more medical functions. In some embodiments, the remote communication devices and systems may facilitate remote patient monitoring, teleconsulting, analyzing one or more data inputs to determine a patient event, or any other suitable medical function.

### CROSS-REFERENCE TO RELATED APPLICATIONS

This application claims priority to and the benefit under 35 U.S.C. § 119(e) of U.S. provisional patent application No. 63/627,402, filed on Jan. 31, 2024 under Attorney Docket No. A1431.70000US00, and titled “SYSTEM AND APPARATUS FOR REMOTE MONITORING AND COMMUNICATION,” for which the entire contents are hereby incorporated by reference herein.

### BACKGROUND

There are many systems for performing remote monitoring and communication in various environments including general-purpose computers to more specialized systems for performing monitoring and communication functions.

### SUMMARY

As remote and hybrid work environments have become increasingly abundant, both within and across industries, the different use cases for teleconferencing, remote monitoring, and other related remote work technologies have expanded. As such, it may be beneficial to provide a communication device that can integrate the many different functions and adapt to the requirements of the user of the user's environment. To support this adaptability, the communication device may be capable of connecting to a variety of different devices that have different functions or are made by different manufacturers and be able to support and control those different devices. Some environments may benefit from fast processing and analysis times so the device may be capable of processing and analyzing data on the device rather than having to transmit the data to some remote device for processing.

In some aspects, it is appreciated that some applications may benefit by performing Artificial Intelligence (AI) and machine learning analysis functions that process collected data. In some conventional systems, the collected data needs to be transmitted to capable computers that are adapted to perform such analyses. It is appreciated that the data collected and transmitted may be significant and there may be delays in transmitting and processing such data. According to some embodiments described herein, it is appreciated that depending on the application, it may be helpful to provide a device that can be installed nearer to a source of data (e.g., images, video, etc.) for the purpose of processing that data more efficiently and timely. The device may permit, for example, operation in a networked environment so that bandwidth consumed for transmission of such data is reduced when implementing many devices.

To this end, a device may be provided that includes certain AI hardware or other machine learning supportive hardware to perform AI functions at the periphery of the network. In some implementations, this device may also be adapted to perform networking functions for connected components used to capture and/or process data at the periphery (e.g., by AI/ML models). According to some embodiments, the device may be capable of collecting and analyzing the data, performing functions responsive to the data and communicating the data to one or more systems.

It is also appreciated that it would be beneficial to have a device capable of adapting to the hardware in the environment in which the device is installed instead of having to rewire the location to support the functionality and adaptability of the device. For instance, in some implementations, it may be desirable that the device supplies power to the devices it may be connected to in addition to communicating and controlling those devices. As discussed above, some of those devices may produce data that can be processed on the device such as video information from a camera or data from another sensor type. In a practical example, the device may be capable of being installed in a patient's hospital room where it can be connected to a television monitor, camera, among other devices, and is capable of controlling, configuring, and handling the communication to/from such devices. For some devices such as sensors, the communication device provides power and configuration control functions.

In some implementations, the devices described herein may be installed in a medical environment where patients are monitored using remote sensors such as cameras and other sensor types. As such, it may be desirable to support one or more remote monitoring functions. For example, the device may be used to support remote conferencing between different specialists, monitor a patient's status while removing any particular identifying information, process and display a patient's health data such as heartrate and blood pressure, and/or determine whether the patient is at risk of falling or at risk of a health event as indicated for example by a heart rate spike, or other functions. The device may also be used to provide data that feeds logistical support functions such as predicting a patient's length of stay, coordinating resource availability, and other supporting functions. As such, according to some embodiments, a flexible device and system is provided for facilitating and adapting to remote communication requirements in a variety of environments.

In some aspects, a device and system for providing flexible remote communication functions may be provided. In some examples, a remote communication device may have a plurality of network interfaces, each network interface configured to connect with a respective external device. To facilitate flexibility, the network interfaces may include, for example, universal serial bus (USB) ports, USB-C ports, USB micro-B ports, Ethernet ports, high-definition multimedia interface (HDMI) ports, or any other suitable network interface, or any combination thereof. The remote communication device may further include one or more network communication processors configured to communicate data with the respective external devices. The data communicated with the external devices may include information, software, firmware, and other data associated with operating, controlling, and updating the external devices.

In some examples, the remote communication device may further include components configured to execute one or more machine learning models capable of processing data received from one or more of the external devices. The component may include conventional processors, machine learning specific processors, for example artificial intelligence (AI) chips, or any other suitable component, or a combination thereof. To further support the flexible nature of the remote communication device, in some examples, the remote communication device may further include other components such as infrared (IR) transceivers for communicating with devices using IR, light emitting diode (LED) indicators, and may store or be configured to receive software for facilitating remote communication functions.

In some aspects, it is appreciated that a system and device for providing flexible remote communication capabilities may be beneficial to allow for a flexible and adaptable remote communication system for various environments. With remote communication being more commonplace, it would be helpful to provide a system that can facilitate the remote communication needs of any one of a multitude of contexts and environments. For example, the system capabilities required to support remote communication in a medical environment may be different than the capabilities required to support remote communication in a corporate environment. As such, the remote communication devices and systems described herein may flexibly facilitate different remote communication functions including, but not limited to teleconferencing and telemonitoring, while minimizing the additional hardware that may be required by supporting on device processing of data used to support the remote communication functions.

According to some aspects, a remote communication device for facilitating communication between remote users is provided. The remote communication device comprises: a plurality of network interfaces, each network interface configured to connect with a respective external device of one or more external devices; one or more network communication processors, configured to communicate data with the one or more external devices connected to the plurality of network interfaces; one or more machine learning models configured to process one or more data inputs received from the one or more external devices.

In some embodiments, the device further comprises a memory unit configured to store data received from the one or more external devices. In some embodiments, at least one of the one or more machine learning models is configured to: receive data from the one or more external devices; determine an indication of whether an event has occurred based at least in part on the received data; and output the indication of whether the event has occurred.

In some embodiments, at least one of the one or more external devices is a camera; and determining an indication of whether the event has occurred comprises: determining whether a person is within a frame of the camera. In some embodiments, determining an indication of whether the event has occurred further comprises: determining whether the person within the frame of the camera is at risk of falling. In some embodiments, determining an indication of whether the event has occurred further comprises: determining whether the person within the frame of the camera has fallen.

In some embodiments, at least one of the one or more external devices is configured to gather health data associated with a patient; the received data is the health data associated with the patient; and determining an indication of whether the event has occurred comprises: determining whether the patient has had a health event based at least in part on the health data associated with the patient. In some embodiments, the one or more machine learning models is further configured to remove identifying features of the person determined to be within the frame of the camera.

In some embodiments, at least one network interface of the plurality of network interfaces is configured to connect to a power source. In some embodiments, the at least one network interface configured to connect to a power source is an ethernet port configured to receive power from the power source using a power-over-ethernet (POE) standard. In some embodiments, the at least one network interface configured to connect to a power source is a universal serial bus (USB) micro-B port configured to receive power from the power source. In some embodiments, each network interface of the plurality of network interfaces is further configured to deliver power to the respective external devices. In some embodiments, at least a subset of the plurality of network interfaces are ethernet ports configured to deliver power and transmit data to the respective external devices using a power-over-ethernet (POE) standard. In some embodiments, at least a subset of the plurality of network interfaces are universal serial bus (USB) ports configured to deliver power and transmit data to the respective external devices.

In some embodiments, the one or more network communication processors are further configured to: receive from a management station, software for controlling or updating firmware of the one or more external devices; control each of the respective external devices of the one or more external devices; and update the firmware of the one or more external devices. In some embodiments, controlling each external device of the one or more external devices comprises: determining one or more methods associated with controlling the external device from the received software; and executing, using the one or more network communication processors, at least one of the one or more methods associated with controlling the external device.

In some embodiments, determining one or more methods associated with controlling the external device from the received software comprises: executing one or more test methods associated with controlling the one or more external devices; and determining which of the one or more test methods control the external device. In some embodiments, the one or more network communication processors are further configured to transmit data associated with the device and the one or more external devices to the management station.

In some embodiments, the device further comprises an infrared (IR) transmitter; at least one of the one or more external devices comprises a display having an IR receiver; and the one or more network communication processors are further configured to control the display by causing the IR transmitter to transmit an IR signal to the IR receiver of the display.

In some embodiments, at least one of the one or more external devices is a camera; at least one of the one or more external devices is a microphone; and the one or more network communication processors are further configured to: transmit first data collected by the camera and the microphone across a network to a receiving device configured to output the first data; receive second data collected by a second camera and a second microphone associated with the receiving device; and output the second data.

In some embodiments, the first data comprises a plurality of frames and transmitting the first data comprises transmitting each frame of the plurality of frames; and the one or more network communications processors are configured to: determine a dropped frame rate indicative of a number of frames of the plurality of frames that were not outputted by the receiving device; and adjust a resolution of the first data until the dropped frame rate is below a threshold dropped frame rate.

In some embodiments, the first data comprises a plurality of frames and transmitting the first data comprises transmitting each frame of the plurality of frames; and the one or more network communications processors are configured to: determine a dropped frame rate indicative of a number of frames of the plurality of frames that were not outputted by the receiving device; and adjust a resolution of the first data until the dropped frame rate is within a threshold dropped frame rate range.

According to some aspects, a method is provided. The method comprises: receiving, with a remote communication device at a first location, data from one or more external devices operatively coupled with the remote communication device; determining, using at least one processor executing one or more machine learning models, one or more outputs associated with an event occurring at the first location by providing the data from at least one external device of the one or more external devices as input to the machine learning model; and transmitting the one or more outputs to a management station over a communication network.

In some embodiments, at least one of the one or more external devices is a camera; the first location is a patient room within a medical facility; and determining one or more outputs associated with an event occurring at the first location comprises determining whether the patient room is occupied by a patient by: providing image data received from the camera to the one or more machine learning models; and determining, using the one or more machine learning models, one or more features of the patient room indicative of whether the patient room is occupied.

In some embodiments, determining one or more features of the patient room indicative of whether the patient room is occupied comprises: determining whether the patient is within a frame of the camera based at least in part on the image data. In some embodiments, determining one or more features of the patient room indicative of whether the patient room is occupied comprises: determining a presence in the patient room of at least one of: personal belongings of the patient, rumpled or missing sheets on a bed in the patient room, and/or one or more visitors in the patient room. In some embodiments, the method further comprises determining a fall risk of the patient occupying the patient room by providing the received image data as input to a second machine learning model of the one or more machine learning models.

In some embodiments, receiving data from one or more external devices comprises receiving health data associated with a patient from at least one external device of the one or more external devices configured to gather health data. In some embodiments, determining one or more outputs associated with an event at the first location comprises determining whether the patient has had a health event based at least in part on the health data associated with the patient.

In some embodiments, at least one of the one or more external devices is a camera; the first location is an operating room within a medical facility; and determining one or more outputs associated with an event occurring at the first location comprises: identifying, using the one or more machine learning models, one or more features indicative of an identity of a patient in the operating room in image data received from the camera; and removing the one or more features indicative of an identity of a patient in the operating room from the image data.

According to some aspects, a system for facilitating teleconferencing between remote users is provided. The system comprises: a management station; a remote communication device in communication with the management station, the remote communication device comprising: one or more network interfaces, at least a subset of the network interfaces configured to connect the remote communication device to a respective external device of one or more external devices; and one or more processors configured to: transmit information associated with the remote communication device to the management station; receive from the management station, a subset of methods of a plurality of methods for controlling or updating the one or more external devices; control the one or more external devices; and transmit information associated with the one or more external devices.

In some embodiments, the management station comprises a display configured to display the information associated with the remote communication device and the information associated with the one or more external devices in a user interface. In some embodiments, the remote communication device is a first remote communication device of a plurality of remote communication devices; and the management station is in communication with each of the plurality of remote communication devices.

In some embodiments, each remote communication device of at least a subset of remote communication devices of the plurality of remote communication devices is connected with a respective camera configured to collect image data; and the management station is configured to receive the collected image data from the subset of remote communication devices and display on a display, the collected image data from at least a subset of the plurality of remote communication devices.

In some embodiments, the remote communication device is configured to receive from the management station, a subset of methods of a plurality of methods for controlling or updating the one or more external devices by: determining, with the one or more processors of the remote communication device, a manufacturer of at least one external device of the one or more external devices; determining a subset of methods of the plurality of methods associated with the manufacturer of the at least one external device of the one or more external devices; and transmitting, with the management station, the subset of methods associated with the manufacturer of the at least one external device of the one or more external devices to the one or more processors of the remote communication device. In some embodiments, the remote communication device is configured to control the one or more external devices by: executing at least one method of the subset of methods associated with the manufacturer of the at least one external device of the one or more external devices.

In some embodiments, the remote communication device is configured to receive from the management station, a subset of methods of a plurality of methods for controlling or updating the one or more external devices by: receiving test methods associated with controlling the one or more external devices; executing the test methods associated with controlling the one or more external devices; determining which test methods control at least one external device of the one or more external devices; and receiving from the management station one or more methods associated with the test method determined to control at least one external device of the one or more external devices.

In some embodiments, at least one network interface of the plurality of network interfaces is configured to connect to a power source. In some embodiments, the at least one network interface of the plurality is a universal serial bus (USB) micro-B port configured to receive power from the power source via a USB cable. In some embodiments, the at least one network interface of the plurality is configured to receive power from a power source and facilitate transmission of data between the remote communication device and the management station using a power-over-ethernet (POE) standard.

According to some aspects, a method is provided. The method comprises: receiving, from a remote communication device, information associated with the remote communication device to a management station, the information include device information associated with one or more external devices operatively coupled to the remote communication device; transmitting, to the remote communication device, a subset of methods of a plurality of methods for controlling or updating the one or more external devices from the management station.

In some aspects, the method further comprises: displaying, with a display of the management station, information associated with the remote communication device and the information associated with the one or more external devices in a user interface. In some embodiments, the remote communication device is a first remote communication device of a plurality of remote communication devices in communication with the management station; the method further comprising: displaying, in a first user interface, information associated with the plurality of remote communication devices and information associated with respective one or more external devices operatively coupled with each remote communication device of the plurality of remote communication devices.

In some embodiments, the method further comprises: receiving image data from each remote communication device, the image data being received by each remote communication device from at least one external device operatively coupled with the respective remote communication device; and displaying the image data on a display of the management station in a second user interface.

In some embodiments, transmitting, to the remote communication device, a subset of methods of a plurality of methods for controlling or updating the one or more external devices comprises: determining a manufacturer of at least one external device of the one or more external devices; determining a subset of methods of the plurality of methods associated with the manufacturer of the at least one external device of the one or more external devices; and transmitting, from the management station, the subset of methods associated with the manufacturer of the at least one external device of the one or more external devices to the remote communication device.

In some embodiments, the method further comprises: controlling, with the remote communication device, the one or more external devices based on the transmitted subset of methods. In some embodiments, controlling the one or more external devices comprises: executing at least one method of the subset of methods associated with the manufacturer of the at least one external device of the one or more external devices.

In some embodiments, transmitting, to the remote communication device, a subset of methods of a plurality of methods for controlling or updating the one or more external devices comprises: transmitting test methods associated with controlling the one or more external devices. In some embodiments, the method further comprises: executing, using the remote communication device, the test methods associated with controlling the one or more external devices; determining which test methods control at least one external device of the one or more external devices; and transmitting, from the management station, one or more methods associated with the test method determined to control at least one external device of the one or more external devices. In some embodiments, controlling the one or more external devices comprises: executing at least one method of the subset of methods associated with the test method determined to control at least one external device of the one or more external devices.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 shows an exemplary device, according to some embodiments.

FIG. 2 shows an exemplary remote communication device with external devices connected to the remote communication device via the network interfaces, according to some embodiments.

FIG. 3 shows an exemplary embodiment of a remote communication device having a wireless interface with a connected display, according to some embodiments.

FIG. 4 shows a diagram of a component implementing a model configured to process data received from one or more external devices connected to the remote communication device, according to some embodiments.

FIG. 5 shows an exemplary remote communication system having a remote communication device connected to external devices and a management station, according to some embodiments.

FIG. 6 shows an example user interface of a management station for displaying information regarding connected devices, according to some embodiments.

FIG. 7A shows an example of a management station receiving from a remote communication device data obtained from an external device and outputting the received data, according to some embodiments.

FIG. 7B shows an example of a management station receiving from a plurality of remote communication devices data obtained from a plurality of external devices and outputting the received data, according to some embodiments.

FIG. 8A shows an example method for optimizing network bandwidth usage of a remote communication device during a remote communication session, according to some embodiments.

FIG. 8B shows an example method for optimizing network bandwidth usage of a remote communication device during a remote communication session, according to some embodiments.

FIG. 9A shows an example method for controlling one or more external devices connected to a remote communication device.

FIG. 9B shows an example method of determining a set of software methods associated with controlling or updating an external device, according to some embodiments.

FIG. 9C shows an example method of determining a set of software methods associated with controlling or updating an external device, according to some embodiments.

### DETAILED DESCRIPTION

As discussed above, it may be helpful to provide a flexible system and apparatus for facilitating and meeting various requirements for performing remote monitoring and communications in a variety of environments. As remote and hybrid work environments have become increasingly abundant, both within and across industries, the different use cases for teleconferencing, remote monitoring, and other related remote work technologies have expanded. This has allowed for greater flexibility and accessibility to different resources and personnel across industries. For example, medical teams in one hospital may have access to a greater variety of specialists located in geographically distributed locations to help address their patients' needs, or patients with limited mobility may access remote healthcare appoints (telehealth) from their own home.

However, as the use of remote work technologies has expanded, it can be appreciated that the requirements for remote communication devices, software, and systems have similarly expanded as different industries and environments have adopted remote or hybrid work models or have put in place new procedures based on remote communication capabilities. For example, it can be appreciated that a medical environment may have substantially different hardware or functional capabilities than a corporate environment. These differences may span different categories, for example, physical capabilities, occupational capabilities, or any other capability that an environment may have or lack. As discussed with respect to the examples below, it can be further appreciated that different situations in the same environment may similarly have different capabilities. For example, a tele-consult occurring during a surgical procedure may have camera with a higher resolution or may utilize more network bandwidth than monitoring a patient in their own hospital room. As such, the remote communication devices and systems described herein may flexibly facilitate different remote communication functions including, but not limited to teleconferencing and telemonitoring, while minimizing the additional hardware that may be used to support the communication functions.

For example, some environments may benefit from fast processing and analysis times so the device may be capable of processing and analyzing data on the device rather than having to transmit the data to some remote device for processing. It can be appreciated that some applications may benefit by performing Artificial Intelligence (AI) and machine learning analysis functions that process collected data so a device may be provided that includes certain AI hardware or other machine learning supportive hardware to perform AI functions at the periphery of the network. It is also appreciated that it would be beneficial to have a device capable of adapting to the hardware in the environment in which the device is installed instead of having to rewire the location to support the functionality and adaptability of the device. In some implementations, the devices described herein may be installed in a medical environment where patients are monitored using remote sensors such as cameras and other sensor types. As such, it may be desirable to support one or more remote monitoring functions. Details of these examples will be discussed further herein.

As further discussed above, a device and system for providing flexible remote communication functions may be provided. In some examples, a remote communication device may have a plurality of network interfaces, each network interface configured to connect with a respective external device. To facilitate flexibility, the network interfaces may include universal serial bus (USB) ports, USB-C ports, USB micro-B ports, Ethernet ports, high definition multimedia interface (HDMI) ports, or any other suitable network interface, or any combination thereof. The remote communication device may further include one or more network communication processors configured to communicate data with the respective external devices. The data communicated with the external devices may include information, software, firmware, and other data associated with operating, controlling, and updating the external devices. In some examples, the remote communication device may further include components configured to execute one or more machine learning models capable of processing data received from one or more of the external devices. The component may include conventional processors, machine learning specific processors, for example artificial intelligence (AI) chips, or any other suitable component, or a combination thereof. To further support the flexible nature of the remote communication device, in some examples, the remote communication device may further include other components such as infrared (IR) transceivers for communicating with devices using IR, light emitting diode (LED) indicators, and may store or be configured to receive software for facilitating remote communication functions. Details of remote communication devices, systems, and methods will be discussed in more detail below.

Accordingly, the devices and systems are provided for supporting and facilitating a flexible remote communication network that can be used in a variety of environments. Described herein are various techniques, including systems, computerized methods, and non-transitory instructions, that facilitate a flexible remote communication device that can adapt to the environment that it is being used in. According to some embodiments, a remote communication device may be provided to facilitate and support remote communication in a variety of different environments. For example, the remote communication device may be configured to facilitate teleconferencing functions by transferring video and audio signals, or other suitable signals, to a remote device over a communication network. In some examples, the remote device may be a second remote communication device as described herein or may be any other suitable device that may support teleconferencing capabilities. In some examples, the remote communication device may include a plurality of network interfaces. At least one of the network interfaces may be connected to a power source for providing power to the remote communication device. In some examples, the network interface configured to be connected with the power source may be an Ethernet port to receive both power and data according to a power-over-ethernet (POE) standard or a POE+ or POE++ standard. In other examples, some environments may not support POE standards and the power port may be a USB micro-B, USB-C, or any other suitable port for providing power.

The remote communication device may further include one or more network interfaces to operatively connect with any number of different devices in one or more modalities to allow for flexibility in specific hardware able to connect with the remote communication device. For example, the remote communication device may have one or more network interfaces to facilitate a wired connection between the remote communication device and an external device(s) such as a television, monitor, microphone, camera, health monitoring device (e.g., heart monitor, blood pressure monitor, etc.) or other suitable external device. The one or more network interfaces may include one or more ethernet ports, USB ports, USB-C ports, or any other suitable port or any combination thereof. Being a wired connection, both data and power may be supplied to the external device(s) using the wired connection, for example, following power-over-ethernet (POE) or other suitable protocol. Additionally or alternatively, the external devices may receive only one of data or power from the remote communication device through the wired connection. For example, an external device may be connected to a separate power source and thus, may only receive data through the wired connection with the remote communication device. In other examples, one or more of the external devices may be communicatively coupled with the remote communication device through a wireless connection using a Wi-Fi protocol, infrared (IR) signaling, Bluetooth or any other suitable wireless connection. The connected external devices may be hardware required for certain remote communication capabilities. For example, one or more of the external devices may include one or more of cameras, microphones, speakers, displays. In some examples, the external devices may additionally or alternatively include monitoring devices or measurement devices. The details of the potential external devices will be described further below.

### Device Agnosticism

The inventors have recognized and appreciated that different environments may have access to different hardware components that do primarily the same function. For example, one location may use cameras produced by a first manufacturer whereas a second location may use cameras produced by a second manufacturer. While performing the same function, the two different cameras may be controlled differently or may respond to different control methods. Accordingly, the inventors have developed techniques and methods to allow for the remote communication devices described herein to remain agnostic to the specific external devices that may be connected to them. For example, the network interfaces may be configured to support a number of different connection protocols as described above and may include a combination of types of ports such as ethernet ports, USB ports, USB-C ports, high-definition multimedia interface (HDMI) ports or other suitable ports so that any suitable piece of external hardware may be connected to the device through a wired connection and may further include a combination of different components to wirelessly communicate with the external devices via wireless communication methods such as WiFi, IR, Bluetooth, or any other suitable wireless communication method. This device capability may be useful, for example, for extending monitoring/teleconferencing capabilities to remote locations where the external devices may include different combinations of devices. For example, the device may be installed in home for telemonitoring purposes, a patient room, a surgery room, or other location where the external device configuration, power requirements and capabilities differ widely.

In some examples, the remote communication device may further include one or more processors for controlling at least one of the external devices, updating the firmware of at least one of the external devices, and processing data received from at least one of the external devices. The inventors have recognized and appreciated that in addition to different hardware compatibility requirements, different external devices may have different software compatibility requirements and may require different software methods for controlling the external device. As such, the remote communication device may include a memory unit configured to store software methods for controlling any number of external devices and the one or more processors may further be configured to determine which of the software methods may be used to control different external devices. In some examples, rather than storing all of the methods for controlling any number of external devices, the methods may be stored at a management station communicatively coupled with the remote communication device and the remote communication device may be configured to receive from the management station and store only those methods associated with controlling the external devices that are connected to the remote communication device.

In some examples, a subset of the software methods may be associated with a manufacturer of an external device. Determining which of the software methods may be used to control an external device may thus include determining the manufacturer of a particular external device. The one or more processors may then determine a subset of the software methods that is associated with the manufacturer of the external device. For example, it may be known that an external device produced by a particular manufacturer may use a set of standard control methods for controlling the external device, for example, an external device may be an Internet Protocol (IP) based device which may implement Open Network Video Interface Forum (ONVIF) control standards, Session Initiation Protocol (SIP) control standards, USB video class (UVC) control standards, HDMI Consumer Electronics Control (HDMI CEC) control standards or any other suitable control standards. Alternatively or additionally, it may be known that an external device produced by a particular manufacturer may use a set of control methods specific and specialized to that particular external device. In examples where the methods are stored at a management station, the remote communication device may then receive only the subset of software methods that is associated with the manufacturer of the external device.

In some examples, manufacturer information of an external device may not be available, or a subset of software methods may not be associated with a particular manufacturer or methods outside of the subset of software methods associated with the manufacturer may be capable of controlling the external device in addition to the subset of software associated with the manufacturer. Determining which of the software methods may be used to control an external device may include executing one or more test methods associated with controlling the external device. In some examples, the one or more test methods may be received from the management station. Determining which of the software methods may be used to control the external device may then include determining which of the one or more test methods are compatible with the external device. A subset of methods of the software methods for controlling an external device may be associated with the one or more test methods determined to be compatible with the external device. The subset of methods may then be determined to also be compatible with the external device. In some examples, the remote communication device may then receive the subset methods associated with the test method determined to be compatible with the external device.

After determining and/or receiving the subset of methods as described in the examples above, the one or more processors may control the one or more external devices using the subset of methods determined to be compatible with the one or more external devices. For example, the one or more processors may control a first external device by executing one or more methods of the subset of methods determined to be compatible with the first external device and may control a second external device by executing a different one or more methods of the subset of methods determined to be compatible with the second external device. The examples described above may be further used in the same manner to determine software associated with updating the firmware of one or more external devices. In some examples, the software associated with updating the firmware of one or more external devices may include a firmware update for a first external device of the external devices. The one or more processors may update the firmware of the first external device by executing the firmware update of the software associated with updating the firmware of the first external device and causing the first external device to implement the firmware update.

### On Device Storage and Processing

The inventors have further recognized and appreciated that it may be beneficial to additionally store and process information and data directly on a remote communication device in certain environments. For example, storing and processing the data and information on device may reduce the bandwidth required for storing and processing the data and result in faster processing outputs, which may be critical in certain environments. Accordingly, the inventors have developed techniques for facilitating on-device processing on a remote communication device or as part of a remote communication system.

In some examples, a remote communication device may have one or more components configured to process one or more types of data received from one or more external devices that may be connected to the remote communication device. In some examples, the one or more components may include conventional processors. In some examples, the one or more components may include artificial intelligence (AI) chips, or may include a combination of conventional processors and AI chips. The one or more components, for example, AI chips may implement a machine learning model to perform any of the methods described below. For example, an AI chip may implement a large language model (LLM), neural network, convolutional neural network, recurrent neural network, or other suitable machine learning model. In some embodiments, the one or more components may implement more than one machine learning models to perform one or more of the various methods described herein. For example, an LLM may be implemented to make predictions based on various written records and a computer vision model may be implemented to detect various events. In some examples, multiple LLMs or computer vision models may be used. For example, a first computer vision model may be implemented for a first use, for example, detecting whether a person fell within a frame of a camera and a second computer vision model may be used to determine whether there is any identifying information about a patient within a frame of a camera. The computer vision models may include object detection models, classification, body pose estimation models, segmentation models or any other suitable computer vision models. Additionally or alternatively, the one or more components may implement voice recognition models alone or in connection with the other models described herein.

The one or more components may be configured to process one or more types of data, for example, image data, audio data, health data, or any other suitable type of data, or a combination thereof. In some examples, the one or more components may process image data received from a camera that is connected to the remote communication device. Processing the image data may include determining a subject or content of the image data, for example, the one or more components may be configured to determine based on the image data whether a person is within a frame of the camera. Determining the subject or content of the image data may be done by any suitable method. Processing the image data may further include determining whether an event has occurred within the frame of the camera. In some examples, the determined event may be that a person within the frame of the camera is at risk of falling or has already fallen. Additionally or alternatively, processing the image data may include altering the image data in some manner. For example, processing the image data may include blurring or otherwise obscuring or removing from the image data, identifying characteristics of a person within the frame of the camera such as the person's face.

In some examples, the received data may be health data obtained from one or more medical devices connected to the remote communication device and processing the health data may include. The determined event may be, for example, an increase in blood pressure determined from data received from a blood pressure monitor, or an irregular heartbeat determined from data received from a heart monitor, or any other suitable health event determined from health data.

The remote communication device may be configured to output the processed data in any suitable manner. In some examples, the remote communication device may output the processed data to a second remote communication device during a remote communication session. In some examples, the remote communication device may output the processed data to a display or other output device connected to the remote communication device through one or more of the network interfaces of the remote communication device and configured to output the processed data. In some examples, the remote communication device may output the processed data to a management station in communication with the remote communication device. In some examples, the remote communication device may not immediately output the processed data and the remote communication device may include one or more memory storage units configured to store either or both of the raw data received from one or more of the external devices and the processed data. The one or more memory storage units may be configured to store the raw and/or processed data for a specific amount of time, for example, a few minutes, or may be configured to store the raw and/or processed data indefinitely.

### Management Station

As discussed above, any of the remote communication devices or other devices described herein may be in communication with a management station either directly or indirectly through the remote communication device, for example, the remote communication device may facilitate data and information transfer from the management station to an external device connected with the remote communication device or from the external device to the management station. The management station may be a stationary station with which various remote communication devices described herein can communicate with. For example, the management station may implemented on one or more computing devices located at a nurses station in a medical facility. In some embodiments, the management station may be a mobile station that can be moved around the medical facility. For example, the management station may be implemented on one or more computing devices located on (or integrated with) a mobile cart (e.g. a cart having wheels). Apart from storing and transferring software methods associated with updating or controlling an external device, as described above and further herein, the management station may be further configured to receive data and information from the remote communication device and/or the external devices that may be connected to the remote communication device.

### Error Monitoring

In some examples, the management station may be configured to receive information about the remote communication device. For example, the management station may receive from the remote communication device, information about the remote communication device including, but not limited to, a name of the device, a location the device may be installed, a status of the device indicating whether the device is online, the internet protocol (IP) address, media access control (MAC) address, what external devices are connected to the remote communication device, or any other information about the remote communication device. The management station may further receive from the remote communication device, information about the external devices that may be connected to the remote communication device through the network interfaces of the remote communication device, for example, the type of external device, the manufacturer of the external device, location that the external device may be installed online status, monitoring status, IP address, MAC address, or any other information about the external devices. The management station may comprise a display that is configured to display the information about the remote communication device and the external devices in a user interface. In some examples, the user interface may include a list of remote communication devices and external devices a display the information about the devices next to the respective device in the list.

In some examples, the management station may be further configured to monitor the function of the remote communication device and the external devices and any errors that may occur with the remote communication device or the external devices. For example, the management station may receive information from the remote communication device that a network interface of the remote communication device has a loose connection, a method that the remote communication device has attempted to execute has failed, whether the external devices are functioning properly, or any other potential error that may occur. In some examples, the upon receiving information from the remote communication device about an error that may have occurred, the management station may be configured to alert a user of the management station, the remote communication device, and/or the external device that an error has occurred. In some examples, the alert may include highlighting or otherwise indicating an error next to the entry for the respective device in the user interface of the management station. In some examples, the alert may include transmitting information about the error to the remote communication device to display the information on a display that may be connected to the remote communication device. In some examples, the alert may include transmitting information about the error to a mobile device of a user. In some examples, the alert may include transmitting information about the error to the provider of the remote communication device and/or the manufacturer of the respective external device.

### Telemonitoring

In some examples, additionally or alternatively to the information described above, the management station may be configured to receive data obtained by one or more of the external devices connected to the remote communication device. In some examples, the received data may be processed data that was obtained by one or more of the external devices and then processed on the remote communication device or the respective external device and then transmitted to the management station. In some examples, the data may be raw data obtained by one or more of the external devices to be processed at the management station. The management station may display using a display, or otherwise output the received data.

For example, one of the external devices may be a camera configured to obtain image data. The camera may transmit the raw image data to the remote communication device. In some examples, the remote communication device may process the image data by any suitable method before transmitting the processed image data to the management station. Additionally or alternatively, the remote communication device may transmit the raw image data to the management station. The management station may then display the received image data on a display of the management station. In some examples, the management station may receive data from more than one external device connected to the same remote communication device or from more than one external device each connected to a separate remote communication device, or a combination thereof, for example, two external devices connected to a first remote communication device and one external device connected to a second remote communication device. The management station may display the received data simultaneously, for example, using a panel associated with each external device or may allow a user to select data from a specific external device.

In some embodiments, the management station may be configured to ensure the privacy of patients in certain circumstances. For example, when a patient is changing or otherwise has an expectation of privacy, the management station may receive an indication as such, and may block the video feed of the particular patient's room. In some embodiments, the determination may be made using a machine learning model configured to identify one or more features indicative of whether a video feed should be cut off to ensure the patient's privacy. Alternatively or additionally, the determination may be made based on input from the patient or medical personnel. For example, in some embodiments, one of the external devices connected to the remote communication device may include an input device (e.g., button, keyboard, microphone), which the patient or a medical personnel may operate to provide input to the remote communication device to cause the remote communication device to cut off a video feed or block the video feed from being viewed at the management station.

### Bandwidth Optimization

The inventors have further recognized and appreciated that different environments, different use cases, and different associated hardware may have an effect on a remote communication network's bandwidth and its ability to facilitate smooth audio and visual data transfer during a remote communication session. For example, certain hardware may only facilitate a certain maximum transfer bandwidth, or a certain environment may have other devices using up more bandwidth, leaving the remote communication device with less network bandwidth to work with. Accordingly, the inventors have developed techniques and methods for optimizing the bandwidth used for audio and visual data being transferred during teleconferencing and telemonitoring and other suitable remote communication uses.

In some examples, a remote communication device may transmit image data and audio data to a device configured to receive the image and audio data over a communication network and output the received data. In some examples, the device configured to receive and output the data may be another remote communication device, an end-user computer, mobile device, or any other suitable device for receiving and outputting the data. The image data may comprise a plurality of frames. The remote communication device may then determine a dropped frame rate. In some examples, the remote communication device may communicate directly with the device configured to receive and output the data to determine the dropped frame rate. For example, the remote communication device may receive from the device a number of frames that have been discarded or dropped between receiving the image data and outputting the image data due to insufficient network bandwidth or any other suitable causes. The remote communication device may determine the dropped frame rate based on the number of frames that have been discarded or dropped between receiving the image data and outputting the image data.

The remote communication device may then compare the determined dropped frame rate with a threshold dropped frame rate. In some examples, the threshold dropped frame rate may be a set threshold. In some examples, the threshold dropped frame rate may be determined during a remote communication session, for example, a teleconferencing session or telemonitoring session. For example, the threshold dropped frame rate may determined based on the hardware used during the remote communication session, the current network usage or network status during the remote communication session, requirements set by a user during the remote communication session, or any other suitable factors for determining the threshold dropped frame rate. In some examples, other metrics may additionally be used to determine the threshold dropped rate including but not limited to, frame loss, data latency, encryption status, retransmissions, audio/video synchronization, and user feedback. In some examples, the threshold dropped frame rate may be a single value or may include a threshold range of values. In comparing the determined dropped frame rate with the threshold dropped frame rate, the remote communication device may adjust the resolution of the image data which may affect the determined dropped frame rate. For example, decreasing the resolution of the image data may also decrease the determined dropped frame rate. The remote communication device may decrease the resolution of the image data until the determined dropped frame rate is below the threshold dropped frame rate. In some examples, this may be done by repeating the steps described above of transmitting the image data, determining the dropped frame rate, and adjusting the resolution. In some examples, this may be done by the remote communication device continuously monitoring the determined dropped frame rate as the resolution is being adjusted. It can be appreciated that there may be cases where the determined dropped frame rate is already below or well below the threshold dropped frame rate. The remote communication device may adjust the resolution of the image data by increasing the resolution until the determined dropped frame rate is close to the threshold dropped frame rate or within the threshold range. Alternatively or additionally, the remote communication device may adjust other feed settings to optimize various metrics of the remote communication session, including bandwidth, compression, and frame rate. In adjusting the various settings, an algorithm implementing the above-described method may learn how the settings affect the various metrics used to determine the threshold dropped frame rate and may adjust various thresholds based on that learning.

### Example Use Cases

The technology discussed herein will be further described with respect to example use cases. It can be appreciated that facilitating a flexible remote communication device and remote communication network can provide a great benefit to the medical field by allowing for increased connectivity and communication between medical specialists and providing increased accessibility to medical care to patients who may have limited mobility or ability to visit a medical office. As such, the remote communication devices, systems, and methods described herein can provide a major benefit to the medical field by providing an integrated and flexible platform to facilitate remote communication functions like teleconferencing, telemonitoring, and processing and transfer of health data between remote medical practitioners. It can be further appreciated that different environments within the medical field may have different hardware and functional requirements as discussed with respect to the examples below.

### Patient Room Use

In one example, the remote communication device may be installed in a patient room and may be connected to one or more external devices through the network interfaces of the remote communication device. For example, the remote communication device may be connected to one or more of displays, cameras, microphones, or medical devices such as a heart monitor or blood pressure monitor. The remote communication device, in addition to facilitating remote communication capabilities, may further be configured to provide monitoring functions to a patient in the patient room. For example, the remote communication device may be in communication (wired or wireless) with one or more external devices to facilitate remote patient monitoring. For example, the remote communication device may provide live photos and/or videos of the patient room to one or more remote monitoring devices (displays, speakers), such as at the management station and/or another external remote monitoring location.

In some examples, the remote communication device may have stored or may receive software from the management station for using, controlling, and updating the displays, cameras, microphones, and medical devices. For example, the remote communication device may store or receive software for controlling motion and/or focus of the cameras or volume of the displays.

The remote communication device may further store or receive software for processing any data that may be received from the one or more external devices. For example, the remote communication device may receive software for processing the data received from one or more external devices to determine whether an event has occurred and may include one or more memory storage units for storing the received data and/or the determination of whether an event has occurred. In the case of a patient room, the event may be whether the patient is at risk of falling, has fallen, is in distress, is having a health event, or any other suitable event.

For example, determining whether a patient is at a risk of falling may include determining whether the patient is within the frame of a camera connected to the remote communication device. The remote communication device may then determine whether the patient is standing or moving towards standing up, for example, the patient has moved from lying down to sitting. The remote communication device may determine whether the patient is at risk of falling based at least in part on image data received from the camera. The remote communication device may additionally use information about the patient to determine whether the patient is at risk of falling, for example, age, medical history information, length of stay, or any other suitable information. Alternatively or additionally, the remote communication device may further use data received from other connected external devices to determine whether the patient is at risk of falling, for example, data received from a blood pressure monitor indicating a drop in blood pressure may indicate a higher likelihood of falling. In some implementations, the device may implement one or more machine learning models capable of processing video, image and/or audio data feeds generated by the external devices to determine whether an event (e.g., a patient fall) has or is likely to occur. Because these models are executed at the periphery of the network rather than capturing, sending and processing data at the remote location, available network bandwidth is preserved and the delay in determining whether an event has occurred is reduced significantly.

In making the determinations described herein, the remote communication device may be further configured to provide automated alarms or patient alerts. In some embodiments, when the remote communication device makes a determination, the remote communication device may provide an indication of the determination to the patient, to the management station, or to any other suitable recipient (e.g., to a nurse located at a remote telemonitoring location). For example, if the remote communication device determines that a patient is at risk of falling, the remote communication device may provide that information to the management station, which may cause the management station to alert an operator of the management station of the patient at risk of falling. The alerts/alarms may be visual or auditory, or a combination thereof. For example, the management station may highlight a particular video feed of the patient's room, or output one or more auditory signals indicating the patient and/or room number of the patient at risk of falling.

In some examples, the one or more components configured to process the received data may further facilitate certain care management functions, for example, estimating the patient's length of stay, or likelihood of relapse or other care management functionality. For example, the remote communication device may receive data related to a patient's medical history and use that received data to make predictions about likelihood of relapse or likelihood that the patient may require certain medical resources like MRI machines or medication. In some embodiments, the data may be received from one or more electronic health records (EHR) stored in a database and received over a communication network.

### Operating Room Use

Alternatively, the remote communication device may be installed in an operating room and may be connected to one or more external devices through the network interfaces of the remote communication device. For example, the remote communication device may be connected to one or more of displays, cameras, microphones, or medical devices such as a heart monitor or blood pressure monitor or a surgical device. The remote communication device may be configured to facilitate intraoperative teleconferencing between the surgical team and a remote specialist. The one or more external devices may thus include overhead cameras and microphones, medical devices, and surgical devices. It can be appreciated that the overhead cameras may not capture the details of what is occurring during the surgery so one or more of the external devices may include a surgical camera, for example, a camera disposed on one of the surgical devices configured to capture image data of the surgery while the surgery is occurring. The remote communication device may be configured to transmit to the remote specialist one or more of image data from the overhead cameras, image data from the surgical cameras, and any other data that may facilitate the teleconferencing with the remote specialist.

The remote communication device may further be configured to store and/or transmit image data to outside persons in a HIPAA compliant manner. For example, surgical video may be transmitted to an outside person during or after a teleconsulting session, for example, to provide digital annotation to the video for consulting or educational purposes. As such, identifying health information may be required to be removed from the surgical video. Accordingly, the remote communication device may be configured to process and store or transmit the surgical video in a HIPAA compliant manner by obscuring or removing any identifying health information, for example, the patient's face may be obscured in the video, or the remote communication device may determine that a patient has identifying tattoos and remove or obscure them from the video.

In some embodiments, the remote communication device may be configured to coordinate the operating room workflow. In some embodiments, the remote communication device may identify whether an operating room is in use or is soon to be in use. For example, the remote communication device may receive visual data from a camera and use a machine learning model to analyze whether the operating room is occupied or soon to be occupied. In some embodiments, the machine learning model may be configured to determine whether the room is occupied or soon to be occupied by identifying a position of a privacy drape in the operating room and/or identifying one or more other features in the operating room indicative of whether the operating room is prepared to receive a patient for surgery (e.g., presence of medical devices, medical personnel). Alternatively or additionally, the remote communication device (or management station) may be configured to aggregate information from multiple remote communication devices to identify the movement of patients towards the operating room. For example, the machine learning model may be configured to identify the progression of a patient's bed towards the operating room based on data provided from cameras associated with one or more of the remote communication devices.

Additionally or alternatively, the remote communication device may be configured to provide other patient services using the techniques, methods, and systems described herein. The remote communication device may facilitate remote nursing, care management, digital whiteboards, patient education services, or other patient services.

### Hospital or Other Medical Facility Use

In some embodiments, the remote communication devices and/or the management station may be used to provide real-time or near real-time views and statistics indicative of various medical facility workflows.

For example, the systems and devices described herein may be used to provide a measure of the medical facility's available versus occupied capacities. In some embodiments, the remote communication devices may receive information indicative of whether a patient room is occupied by one or more patients. In some embodiments, this may be done by providing data received from a camera connected to the remote communication device to a machine learning model configured to identify whether one or more patients are present in the patient room. Alternatively or additional, the machine learning model may be configured to identify one or more features of the patient room indicative of whether a patient occupies the room with or without the patient actually being present in the room (e.g., ruffled sheets, personal belongings).

The management station may aggregate information received from each of the connected remote communication devices to determine the number of occupied versus unoccupied patient rooms as compared to the maximum capacity of the medical facility as a whole, or within a particular patient room. The management station may further utilize additional information in making its determination including, but not limited to, the total number of checked in patients and/or visitors, the maximum capacity of the medical facility, or any other suitable information.

In some embodiments, the systems and devices may provide further functionality and workflow analyses including, but not limited to, facilitating the discharge of patients (e.g., by receiving information from an EHR or input from medical personnel), manage the inventory of particular supplies utilized by the medical facility (e.g., disposable medical equipment, pharmaceuticals), manage the receipt of deliveries at a loading dock of the medical facility, identify locations in the medical facility of any patients, medical personnel, and/or supplies, or any other suitable workflow aspects associated with the medical facility.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

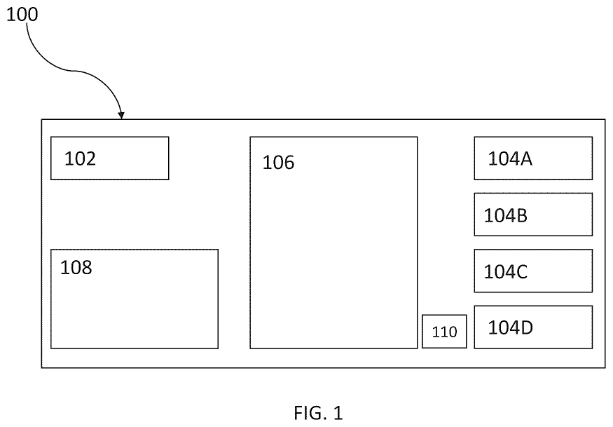

FIG. 1 shows an exemplary remote communication device 100, according to some embodiments. Remote communication device 100 may include a plurality of network interfaces. At least one of the network interfaces may be a power interface 102 configured to connect with a power source to provide power to remote communication device 100. In some embodiments, power interface 102 may be configured to connect with a typical wall outlet. In some embodiments, power interface 102 may be an Ethernet port to receive both power and data according to a power-over-ethernet (POE) standard or a POE+ or POE++ standard. In some embodiments, some environments may not support POE standards and power interface 102 may be a USB micro-B, USB-C, or any other suitable port for providing power. In some embodiments, remote communication device 100 may include more than one power interface 102 of different types, for example, an ethernet port and a USB micro-B port to be able to connect to a power source in different environments or have a redundant power source in case one or the other fails.

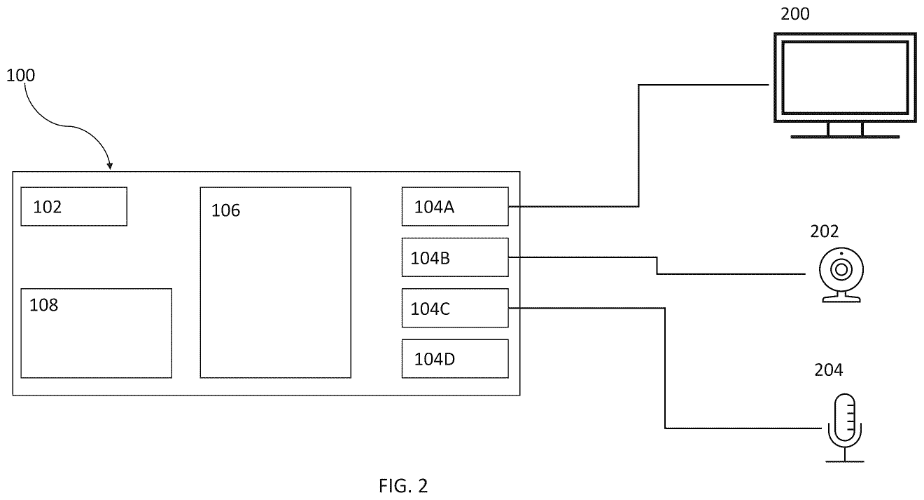

FIG. 2 shows an exemplary remote communication device 100 with external devices connected to the remote communication device via the network interfaces, according to some embodiments. The other network interfaces 104A-104D may be configured to connect to one or more external devices. For example, the one or more external devices may include display 200, camera 202, and microphone 204. Although only three external devices are shown of three types, it can be appreciated that remote communication device 100 may have N network interfaces (104A, 104B, . . . 104N) configured to connect with N external devices which are not limited to displays, cameras, and microphones. For example, the external devices may include other types of devices like medical devices, user input devices (e.g. keyboard, mouse, etc.), and/or any other suitable device.

In some embodiments, network interfaces 104A-104D may be configured to communicatively couple the external devices 200-204 to remote communication device 100 and transmit data between remote communication device 100 and the external devices 200-204. For example, network interface 104A may communicatively couple remote communication device 100 with display 200. Remote communication device 100 may receive information about display 200 including, but not limited to, manufacturer, power status, and operation status, and may transmit image data and other types of data to display 200 through network interface 104A or may execute methods for controlling display 200 through network interface 104A. For example, network interfaces 104A-104D may be USB ports, USB-C ports, Ethernet ports, HDMI ports or any other suitable interface configured to communicatively couple remote communication device 100 with an external device, or may include any combination thereof.

In some embodiments, network interfaces 104A-104D may further be configured to deliver power to one or more of the external devices connected to remote communication device 100 (e.g. display 200, camera 202, microphone 204). In some embodiments, one or more of network interfaces 104A-104D may be Ethernet ports configured to deliver power to one or more external devices over a POE, POE+, or POE++ protocol in addition to transmitting data between remote communication device 100 and the external devices. In some embodiments, one or more of network interfaces 104A-104D may be USB micro-B ports configured to deliver power to the external devices and transmit data between remote communication device 100 and the external devices. In some embodiments, network interfaces 104A-104D may include both Ethernet ports, USB micro-B ports, and/or any other suitable port type for delivering power and transmitting data.

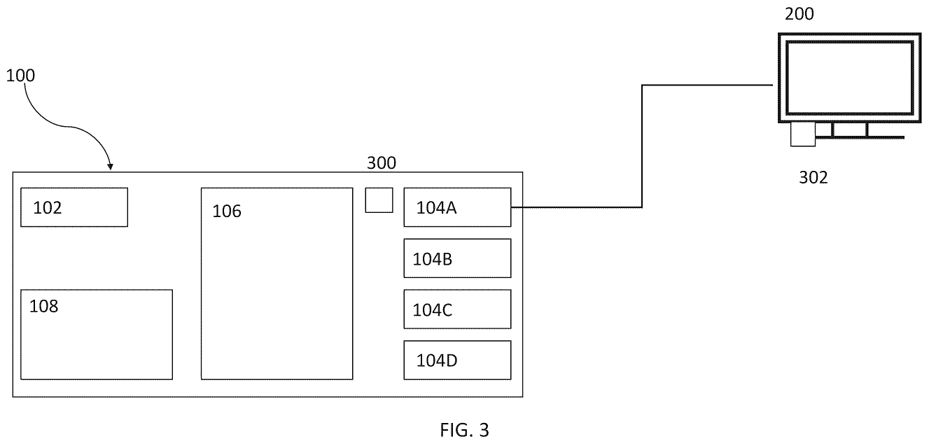

Although wired connections may provide both power and data transmission between remote communication device 100 and the external devices, it can be appreciated that the interface between remote communication device 100 and the external devices may be wireless or may include both a wired and wireless connection. FIG. 3 shows an exemplary embodiment of a remote communication device 100 having a wireless interface with a connected display 200, according to some embodiments. Remote communication device 100 may include wireless transceiver 300 and display 200 may include wireless transceiver 302. Although remote communication device 100 and display 200 are connected with a wired connection via network interface 104A, remote communication device 100 may further communicate with display 200 by sending and receiving signals between wireless transceiver 300 to wireless transceiver 302. For example, wireless transceivers 300 and 302 may be IR transceivers configured to transmit IR signals between remote communication device 100 and display 200. Alternatively or additionally, wireless transceivers 300 and 302 may be configured to communicate wirelessly over any other suitable wireless communication protocol, for example, through WiFi, Bluetooth low energy (BLE), universal asynchronous receiver-transceiver (uART) protocols, etc. While it is shown that display 200 is connected to remote communication device 100 through both wired and wireless means, it can be appreciated that display 200, or any other external device, can be communicatively coupled with remote communication device 100 solely through wireless means or solely through wired means.

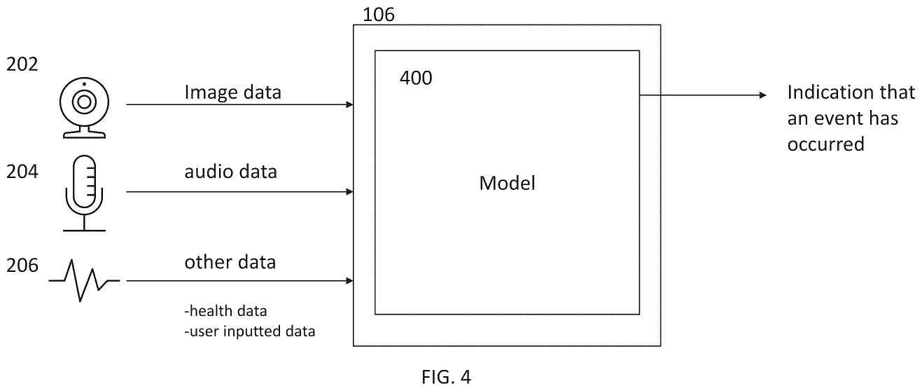

Referring back to FIG. 1, remote communication device 100 may include one or more processors 108 and one or more AI chips 106 configured to process data received from and obtained by the external devices connected to remote communication device 100. The one or more AI chips 106 may be configured to implement one or more models for processing the data received from the external devices to determine whether a particular event has occurred. FIG. 4 shows a diagram of a component (e.g. AI chip 106) implementing a model 400 configured to process data received from one or more external devices 202-206 connected to a remote communication device, according to some embodiments. AI chip 106 may implement a model 400 configured to process one or more sets of data obtained by the external devices connected to a remote communication device (e.g., remote communication device 100). For example, model 400 may be configured to process image data, audio data, health data, or any other suitable set of data, or a combination thereof. Model 400 may be a trained machine learning model, for example, a trained neural network, a large language model (LLM), or any other suitable machine learning model. In some embodiments, model 400 may be an LLM trained on a set of training data comprising text or audio data and trained to perform natural language processing functions. In some embodiments, model 400 may be a computer vision model trained on a set of training data comprising image data and trained to perform computer vision functions. In some embodiments, AI chip 106 may implement both a LLM trained to perform natural language processing functions and a computer vision model trained to perform computer vision functions. AI chip 106 may be any suitable processor capable of implementing AI algorithms and/or trained machine learning models. For example, AI chip 106 may be an m.2 module.

Model 400 may be configured to receive as input, sets of data from one or more external devices connected to the remote communication device that AI chip 106 is disposed upon. For example, model 400 may be configured to receive as input, one or more of image data from camera 202, audio data from microphone 204, and other data from external device 206, which may, for example, be a medical device configured to obtain health data, or a user input device like a keyboard, configured to obtain user inputted data or any other suitable device.

Model 400 may further be configured to determine, based at least in part on the received data, whether an event has occurred and may output an indication when the event has occurred. In some embodiments, model 400 may be a computer vision model configured to determine whether an event has occurred based in part on image data received from camera 202. The event may be whether a person in within the frame of camera 202, whether a person in the frame of camera 202 is at risk of falling or has fallen, or any other suitable event. In some embodiments, model 400 may use multiple types of data to determine whether an event has occurred. For example, model 400 may implement a computer vision model to determine whether a person is within the frame of camera 202 and whether the person is sitting, standing, lying down, moving from lying down to sitting, or from sitting to standing. Model 400 may further implement an LLM configured to receive user inputted text from external device 206, for example, previous medical history notes, which may be used in conjunction with the computer vision determination to determine whether the person within the frame of camera 202 is at risk of falling. Alternatively, model 400 may implement a neural network configured to process health data obtained from external device 206 to determine whether a person within the frame of camera 202 is at risk of falling. For example, external device 206 may be a blood pressure monitor and a drop in blood pressure, in conjunction with the computer vision model determining that the person in the frame of camera 202 is standing up, may indicate that the person is at risk of falling.

It can be appreciated that model 400 or any other model implemented by AI chip 106 may be configured to determine other events. For example, for a device installed in a patient's room in a medical center, model 400 may be configured to make a prediction on the patient's length of stay, what medical resources (e.g., MRI machines, bed types, etc.) a patient may need, readmission risk, or other medical based predictions. Model 400 may make this prediction based on data received from one or more external devices connected to the remote communication device.

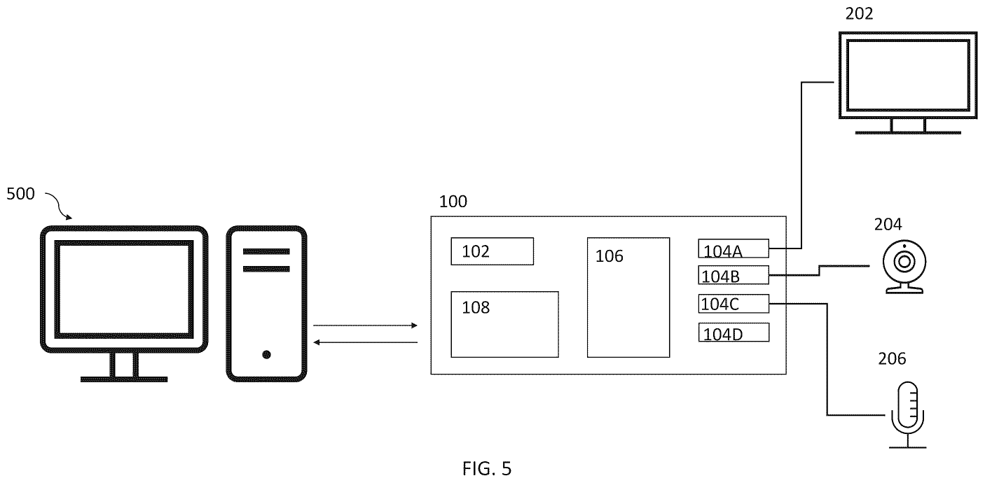

In certain environments, it can be appreciated that any of the remote communication devices described herein may further be part of a network of remote communication devices. As such, it may be beneficial to provide a management station to monitor and facilitate the network of remote communication devices. FIG. 5 shows an exemplary remote communication system having a remote communication device 100 connected to external devices 202-206 and a management station 500, according to some embodiments. Management station 500 may be communicatively coupled with one or more remote communication devices 100 to receive information and data about both remote communication device 100 and the external devices 202-206 that are connected to remote communication device 100. Management station 500 may be configured to receive this information so that it may generally monitor the function of remote communication device 100 and the external devices 202-206. For example, management station 500 may receive from remote communication device 100, information about remote communication device 100 including, but not limited to, a name of remote communication device 100, the location it is installed, the type of device, online status, manufacturer, IP and MAC addressed, and what external devices may be connected. Management station 500 may further receive from remote communication device 100, information about the external devices 202-206 that may be connected to remote communication device 100.

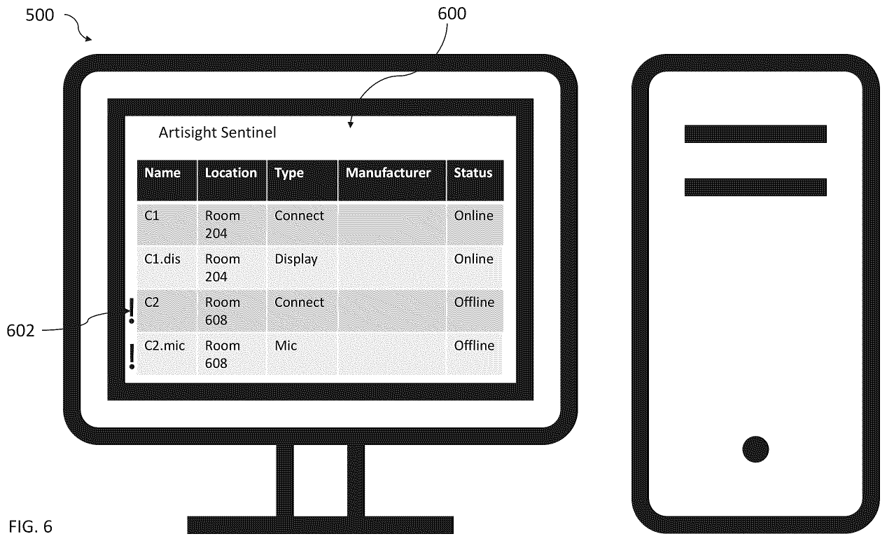

To display all the information received about remote communication device 100 and external devices 202-206, management station 500 may include a display having a user interface for displaying the information. FIG. 6 shows an example user interface 600 of a management station 500 for displaying information regarding connected devices, according to some embodiments. User interface 700 may display the information regarding remote communication device 100 and external devices 202-206 in a list so that a user may see all the information about the connected devices. For ease of reading, user interface 600 may group the devices by remote communication device 100. For example, user interface 600 may display remote communication device 100 and the external devices 202-206 as successive entries in the list and may display a second remote communication device and the external devices connected to it as the next set of successive entries.

In some embodiments, management station 500 may be further configured to monitor the function of remote communication device 100 and the external devices 202-206. In some embodiments, management station 500 may receive from remote communication device 100 an indication that remote communication device 100 and/or one or more of the external device 202-206 may not be functioning properly. For example, the indication may show that an external device may have a loose connection with remote communication device 100, a software method executed by remote communication device 100 has failed, a firmware update of one of the external devices has failed, one of the external devices is not functioning properly, or any other error that may occur. In some embodiments, management station 500 may provide an alert to a user indicating the error that has occurred. For example, management station 500 may display alert symbol 602 next to the device at which the error occurred in user interface 600. In some embodiments, management station 500 may otherwise highlight the dysfunctional device in user interface 600. In some embodiments, management station 500 may cause the alert to be displayed on a display connected to the remote communication device 100 at which the error occurred or may transmit the alert to a mobile application of the user of the dysfunctional device. In some embodiments, management station 500 may transmit the alert to the provider of remote communication device 100 and/or the provider of the dysfunctional device to let them know that an error has occurred.

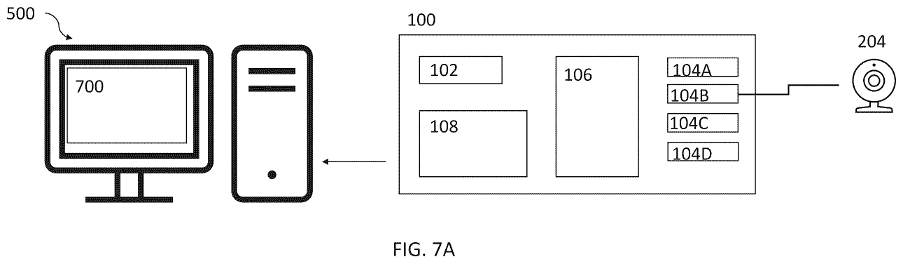

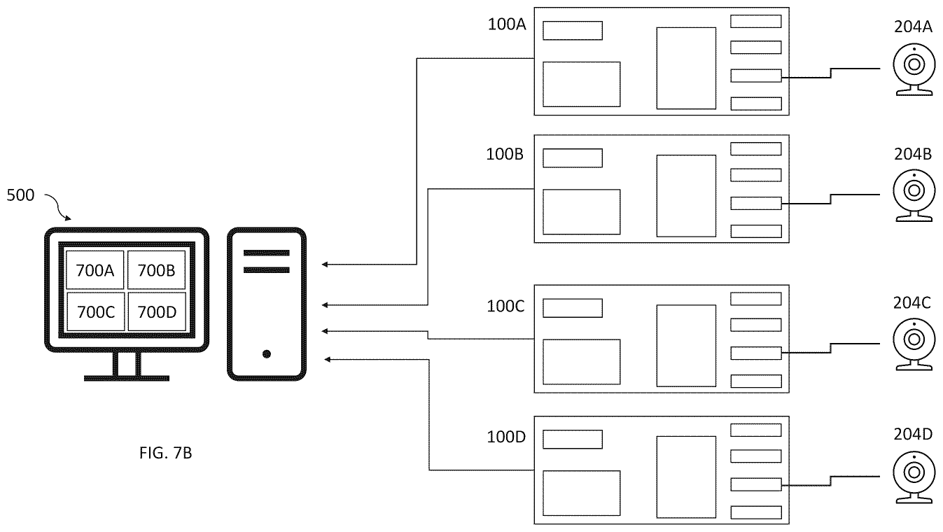

In some embodiments, management station 500 may further be configured to receive and process data obtained by the external devices 202-206. For example, FIG. 7A shows an example of management station 500 receiving, from a remote communication device 100, data obtained from an external device and outputting the received data, according to some embodiments. In the example, the external device is camera 204 configured to obtain and transmit image data. Remote communication device 100 may receive the image data from camera 204 and transmit the image data to management station 500. Management station 500 may display the image data as a video stream 700 or other suitable method. Although image data is used in the example, it is for exemplary purposes only, and the management station 500 may be configured to receive and output any suitable data, for example, by using a speaker to output audio data or may emulate a medical device on a display to display health data.

It can be further appreciated that management station 500 may receive data from more than one external device connected to one or more remote communication devices. FIG. 7B shows an example of management station 500 receiving from a plurality of remote communication devices 100A-100D data obtained from a plurality of external devices 204A-204D and outputting the received data, according to some embodiments. Management station 500 may be communicatively coupled with more than one remote communication device, for example, remote communication devices 100A-100D. Each of remote communication devices 100A-100D may have one or more external devices connected to it, although only one is depicted for each remote communication device for clarity. In some embodiments, the external devices may be cameras 204A-204D each connected to a respective remote communication device 100A-100D and configured to obtain image data. However, it can be appreciated that more than one camera may be connected to a single remote communication device 100 or other types of devices may also be connected to the same remote communication device as any of the cameras. The image data may be transmitted to the management station 500 to be displayed. Management station 500 may display the image data collected from each camera 204A-204D simultaneously, for example, by displaying video streams 700A-700D in a paneled layout. It can be appreciated that management station 500 may not display the image data from each of the cameras simultaneously and may have a user select which data they would like to view. It can be further appreciated that processing the image data may occur at any step in the transfer, for example, on the remote communication devices 100A-100D or at the management station 500.

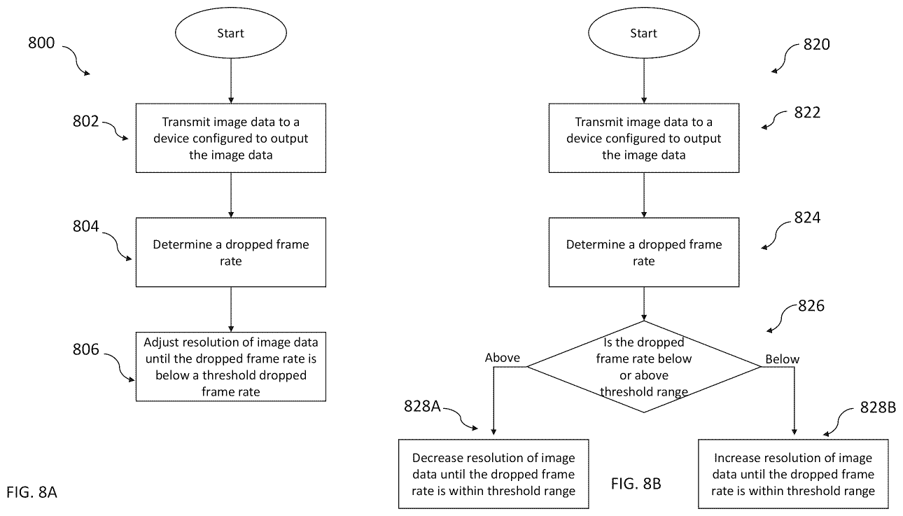

In some environments, the remote communication network may face limitations on network bandwidth of may have to compete with other devices on the network for network bandwidth. In other environments, it may be beneficial to allocate more network bandwidth to transmit higher resolution data or images across the network for certain uses, for example, image data of intricate surgical techniques. FIG. 8A shows an example method 800 for optimizing network bandwidth usage of a remote communication device 100 during a remote communication session, according to some embodiments. Method 800 may start at step 802 with a remote communication device (e.g., remote communication device 100) transmitting image data to a device configured to output the image data. For example, the image data may be transmitted to a second remote communication device on the remote communication network or part of a different remote communication network or may be any other suitable device for outputting the image data.

At step 804, the remote communication device 100 may determine a dropped frame rate comprising the number of frames dropped, discarded, and/or not received by the device out of the total number of frames attempted to be transmitted. In some embodiments, remote communication device 100 may communicate directly with the device it is transmitting the image data to determine the dropped frame rate. For example, remote communication device 100 may detect the number of frames that have been discarded by the device or may receive from the device the number of frames dropped and discarded. Alternatively, remote communication device 100 may determine the number of frames outputted by the device and compare that number of frames to the number of frames attempted to be transmitted.

At step 806, remote communication device 100 may adjust the resolution of the image data until the dropped frame rate is below a threshold dropped frame rate. In some embodiments, remote communication device 100 may compare the determined dropped frame rate with the threshold dropped frame rate. In some embodiments, the threshold dropped frame rate may be determined during a certain remote communication session, for example, based on the hardware being used, the current network usage, user-set requirements, or any other suitable factors. In some embodiments, the threshold dropped frame rate may be predetermined. In some embodiments, the threshold dropped frame rate may be a single value and remote communication device 100 may be configured to process the image data to adjust the resolution of the image data until the dropped frame rate is below the threshold dropped frame rate. In some embodiments, the determined dropped frame rate is monitored continuously as the resolution is being adjusted. In some embodiments, the resolution may be adjusted in discrete steps and the determined dropped frame rate may be determined after the resolution is adjusted each time.

In some embodiments, the threshold dropped frame rate may be a range of values and optimizing the network bandwidth usage may comprise adjusting the resolution of the image data until the determined dropped frame rate is within the range of the threshold dropped frame rate. FIG. 8B shows an example method for optimizing network bandwidth usage of a remote communication device during a remote communication session, according to some embodiments. Steps 822 and 824 are substantially the same as steps 802 and 804 as described above with respect to FIG. 8A. At step 826, remote communication device 100 may determine whether the determined dropped frame rate is above or below the range of the threshold dropped frame rate. If the determined dropped frame rate is above the range of the threshold dropped frame rate, remote communication device 100 may decrease the resolution of the image data until the dropped frame rate is within the range at step 828A. If the determined dropped frame rate is below the range of the threshold dropped frame rate, remote communication device 100 may increase the resolution of the image data until the dropped frame rate is within the range at step 828B. For example, certain circumstances may prioritize resolution over optimal network bandwidth usage and vice versa.

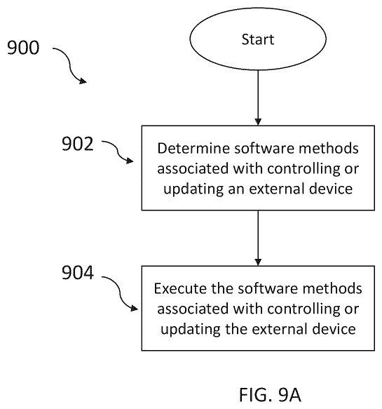

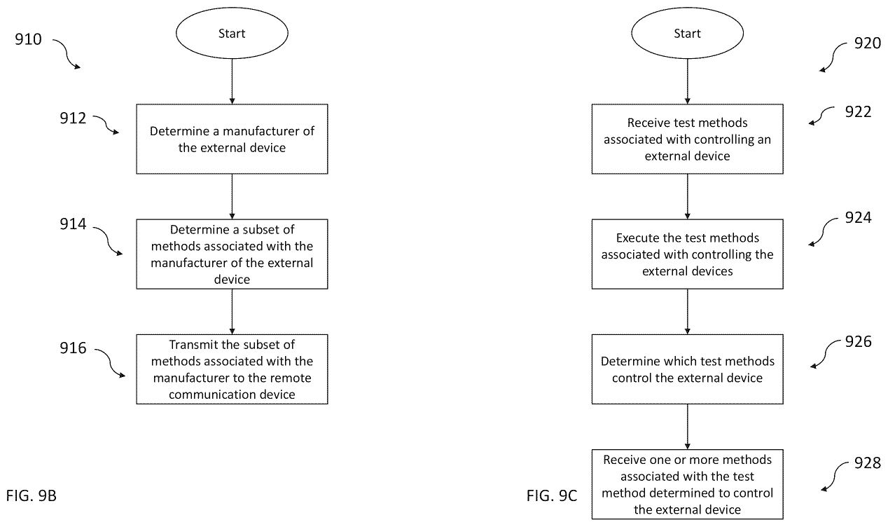

The inventors have recognized and appreciated that different environments may have access to different hardware components that do primarily the same function or have a slightly different function while still maintaining the same primary function. As such, the remote communication devices described herein need a way to control and update the software of any such external device that may be connected to it. One way of doing so is to store or have accessible any software that may be able to control any of the potential external devices. FIG. 9A shows an example method for controlling or updating one or more external devices connected to a remote communication device. Method 900 may be implemented by any of the devices described herein, for example remote communication device 100. Remote communication device 100 may have stored in memory unit 110 a plurality of software methods for controlling or updating any number of potential external devices. Alternatively or additionally, management station 500 may have stored a plurality of software methods for controlling or updating any number of potential external devices. To control or update one of the external devices, remote communication device 100 may determine, at step 902, which software methods of the plurality of software methods are associated with controlling or updating the external device, details of which will be described with respect to FIGS. 9B and 9C below. At step 904, remote communication device 100 may execute the software methods associated with controlling or updating the external device, for example, using one or more processors 108 and transmitting the executed software method through network interface 104.

In some embodiments, a specific subset of software methods of the plurality of software methods may be associated with a specific manufacturer. For example, it may be known that a manufacturer uses control methods that are standard to an industry, for example ONVIF, SIP, UVC control standards for controlling IP based devices, or HDMI CEC control standards for controlling HDMI connected devices. Alternatively or additionally, particular manufacturers may have specialized control methods specific to their devices. As such, determining which software methods are associated with controlling or updating an external device may include determining the manufacturer of the external device. FIG. 9B shows an example method of determining a set of software methods associated with controlling or updating an external device, according to some embodiments. Management station 500 may store one or more software methods for controlling or updating one or more external devices connected to remote communication device 100. At step 912, remote communication device 100 may determine a manufacturer of an external device. At step 914, remote communication device 100 may then determine a subset of methods associated with the manufacturer of the external device. For example, a specific manufacturer may use standard control methods like ONVIF, SIP, or UVC control standards and thus, the subset of methods related to those control methods may be associated with the manufacturer. Alternatively or additionally, a manufacturer may develop specialized control methods and thus, the subset of methods may include the specialized control methods associated with the manufacturer. Management station 500 may then, at step 916, transmit the subset of methods associated with the manufacturer to remote communication device 100. In some embodiments, the software methods may be stored directly on remote communication device 100. Referring back to FIG. 9A, step 904, may thus include executing the subset of methods associated with the manufacturer to control the external device.

In some embodiments, a specific manufacturer may not use standard or specialized control methods, and it may not be known which software methods may control a specific external device. As such, determining which software methods are associated with controlling or updating an external device may include testing different software methods for controlling or updating the external device. FIG. 9C shows an example method of determining a set of software methods associated with controlling or updating an external device, according to some embodiments. At step 912, remote communication device 100 may receive test methods associated with controlling an external device from management station 500. For example, remote communication device 100 may receive different forms of the same function (e.g., control methods) which can then be tested on the external devices. At step 924, the test methods are tested on the external devices by executing the test methods associated with controlling the external devices.

At step 926, remote communication device 100 may determine which test methods control the external device. For example, executing a first test method may result in an error at the external device or the external device may not respond at all. Alternatively, executing a second test method may result in the external device responding to the execution of the test method. Thus, remote communication device 100 may determine that the second test method controls the external device. One or more software methods stored on the management station 500 may be associated with the second test method. At step 928, remote communication device 100 may receive from the management station the one or more software methods associated with the test method determined to control the external device. For example, remote communication device 100 may receive one or more methods associated with the second test method determined to control the external device.

Having thus described several aspects of at least one embodiment of the technology described herein, it is to be appreciated that various alterations, modifications, and improvements will readily occur to those skilled in the art. Such alterations, modifications, and improvements are intended to be part of this disclosure and are intended to be within the spirit and scope of disclosure. Further, though advantages of the technology described herein are indicated, it should be appreciated that not every embodiment of the technology described herein will include every described advantage. Some embodiments may not implement any features described as advantageous herein and in some instances one or more of the described features may be implemented to achieve further embodiments. Accordingly, the foregoing description and drawings are by way of example only.

The above-described embodiments of the technology described herein can be implemented in any of numerous ways. For example, the embodiments may be implemented using hardware, software, or a combination thereof. When implemented in software, the software code can be executed on any suitable processor or collection of processors, whether provided in a single computer or distributed among multiple computers. Such processors may be implemented as integrated circuits, with one or more processors in an integrated circuit module, including commercially available integrated circuit modules known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. However, a processor may be implemented using circuitry in any suitable format.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. In this respect, aspects of the technology described herein may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments described above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the technology as described above. A computer-readable storage medium includes any computer memory configured to store software, for example, the memory of any computing device such as a smart phone, a laptop, a desktop, a rack-mounted computer, or a server (e.g., a server storing software distributed by downloading over a network, such as an app store)). As used herein, the term “computer-readable storage medium” encompasses only a non-transitory computer-readable medium that can be considered to be a manufacture (i.e., article of manufacture) or a machine. Alternatively, or additionally, aspects of the technology described herein may be embodied as a computer readable medium other than a computer-readable storage medium, such as a propagating signal.

The terms “program” or “software” are used herein in a generic sense to refer to any type of computer code or set of processor-executable instructions that can be employed to program a computer or other processor to implement various aspects of the technology as described above. Additionally, it should be appreciated that according to one aspect of this embodiment, one or more computer programs that when executed perform methods of the technology described herein need not reside on a single computer or processor, but the processor functions may be distributed in a modular fashion among a number of different computers or processors to implement various aspects of the technology described herein. Also, various aspects may be implemented using one or more AI/ML models trained on one or more data sets to perform one or more functions, controls, UI operations, etc. responsive to received data.

Computer-executable instructions may be in many forms, such as program modules, executed by one or more computers or other devices. Generally, program modules include routines, programs, objects, modules, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of modules set forth in the foregoing description or illustrated in the drawings. For example, aspects described in one embodiment may be combined in any manner with aspects described in other embodiments.

Also, the technology described herein may be embodied as a method, of which examples are provided herein. The acts performed as part of any of the methods may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A system for facilitating teleconferencing between remote parties, the system comprising:
a management station;
a remote communication device in communication with the management station, the remote communication device comprising:
one or more network interfaces, at least a subset of the network interfaces configured to connect the remote communication device to a respective external device of one or more external devices; and
one or more processors configured to:
transmit information associated with the remote communication device to the management station;
receive from the management station, a subset of methods of a plurality of methods for controlling or updating the one or more external devices;
control the one or more external devices; and
transmit information associated with the one or more external devices.

2. The system of claim 1, wherein:
the management station comprises a display configured to display the information associated with the remote communication device and the information associated with the one or more external devices in a user interface.

3. The system of claim 1, wherein:
the remote communication device is a first remote communication device of a plurality of remote communication devices; and
the management station is in communication with each of the plurality of remote communication devices.

4. The system of claim 3, wherein:
each remote communication device of at least a subset of remote communication devices of the plurality of remote communication devices is connected with a respective camera configured to collect image data; and
the management station is configured to receive the collected image data from the subset of remote communication devices and display on a display, the collected image data from at least a subset of the plurality of remote communication devices.

5. The system of claim 1, wherein:
the remote communication device is configured to receive from the management station, a subset of methods of a plurality of methods for controlling or updating the one or more external devices by:
determining, with the one or more processors of the remote communication device, a manufacturer of at least one external device of the one or more external devices;
determining a subset of methods of the plurality of methods associated with the manufacturer of the at least one external device of the one or more external devices; and
transmitting, with the management station, the subset of methods associated with the manufacturer of the at least one external device of the one or more external devices to the one or more processors of the remote communication device.

6. The system of claim 5, wherein:
the remote communication device is configured to control the one or more external devices by:
executing at least one method of the subset of methods associated with the manufacturer of the at least one external device of the one or more external devices.

7. The system of claim 1, wherein:
the remote communication device is configured to receive from the management station, a subset of methods of a plurality of methods for controlling or updating the one or more external devices by:
receiving test methods associated with controlling the one or more external devices;
executing the test methods associated with controlling the one or more external devices;
determining which test methods control at least one external device of the one or more external devices; and
receiving from the management station one or more methods associated with the test method determined to control at least one external device of the one or more external devices.

8. The system of claim 1, wherein:
at least one network interface of the plurality of network interfaces is configured to connect to a power source.

9. The system of claim 8, wherein:
the at least one network interface of the plurality is a universal serial bus (USB) micro-B port configured to receive power from the power source.

10. The system of claim 8, wherein:
the at least one network interface of the plurality is configured to receive power from a power source and facilitate transmission of data between the remote communication device and the management station using a power-over-ethernet (POE) standard.

11. A method comprising:
receiving, from a remote communication device, information associated with the remote communication device to a management station, the information include device information associated with one or more external devices operatively coupled to the remote communication device;
transmitting, to the remote communication device, a subset of methods of a plurality of methods for controlling or updating the one or more external devices from the management station.

12. The method of claim 11, further comprising:
displaying, with a display of the management station, information associated with the remote communication device and the information associated with the one or more external devices in a user interface.

13. The method of claim 11, wherein:
the remote communication device is a first remote communication device of a plurality of remote communication devices in communication with the management station;
the method further comprising:
displaying, in a first user interface, information associated with the plurality of remote communication devices and information associated with respective one or more external devices operatively coupled with each remote communication device of the plurality of remote communication devices.

14. The method of claim 13, further comprising:
receiving image data from each remote communication device, the image data being received by each remote communication device from at least one external device operatively coupled with the respective remote communication device; and
displaying the image data on a display of the management station in a second user interface.

15. The method of claim 11, wherein:
transmitting, to the remote communication device, a subset of methods of a plurality of methods for controlling or updating the one or more external devices comprises:
determining a manufacturer of at least one external device of the one or more external devices;
determining a subset of methods of the plurality of methods associated with the manufacturer of the at least one external device of the one or more external devices; and
transmitting, from the management station, the subset of methods associated with the manufacturer of the at least one external device of the one or more external devices to the remote communication device.

16. The method of claim 15, further comprising:
controlling, with the remote communication device, the one or more external devices based on the transmitted subset of methods.

17. The method of claim 16, wherein:
controlling the one or more external devices comprises:
executing at least one method of the subset of methods associated with the manufacturer of the at least one external device of the one or more external devices.

18. The method of claim 11, wherein:
transmitting, to the remote communication device, a subset of methods of a plurality of methods for controlling or updating the one or more external devices comprises:
transmitting test methods associated with controlling the one or more external devices.

19. The method of claim 18, further comprising:
executing, using the remote communication device, the test methods associated with controlling the one or more external devices;
determining which test methods control at least one external device of the one or more external devices; and
transmitting, from the management station, one or more methods associated with the test method determined to control at least one external device of the one or more external devices.

20. The method of claim 19, wherein:
controlling the one or more external devices comprises:
executing at least one method of the subset of methods associated with the test method determined to control at least one external device of the one or more external devices.
